(19) United States
(12) Patent Application Publication
(10) Pub. No.: US 2020/0029849 A1
(43) Pub. Date: Jan. 30, 2020

(54) USES OF MINIMALLY INVASIVE SYSTEMS AND METHODS FOR NEUROVASCULAR SIGNAL MANAGEMENT INCLUDING ENDOVASCULAR ELECTROENCEPHALOGRAPHY AND RELATED TECHNIQUES FOR EPILEPSY DETECTION AND TREATMENT

(71) Applicant: INNERVATE MEDICAL, LLC, Coto De Caza, CA (US)

(72) Inventor: Sunil Anil Sheth, Houston, TX (US)

(21) Appl. No.: 16/338,240

(22) PCT Filed: Sep. 29, 2017
(86) PCT No.: PCT/US2017/054448

§ 371 Date: Mar. 29, 2019

Related U.S. Application Data
(60) Provisional application No. 62/401,846, filed on Sep. 29, 2016.

(51) Int. Cl. A61B 5/0478; A61B 5/00; A61N 1/05; A61N 1/36; A61N 1/08

(52) U.S. Cl. CPC A61B 5/0478; A61B 5/6851; A61B 5/6859; A61B 5/6862; A61N 1/086; A61B 5/0031; A61N 1/0534; A61N 1/36064; A61B 5/0022

(57) ABSTRACT

Minimally invasive systems, and tools provided herein in novel enhanced and modified form for this application used has Used deep brain knowledge and structural data to generate novel enhanced intracranial signals to develop, characterize and ameliorate challenges and disease states with application specific tools modified from tradition EEG for Epilepsy to address a myriad of conditions in patients, without any need for invasive protocols traditionally employed. Also disclosed is a novel enhanced system that can be permanently implanted (like a pacemaker) that can both sense epileptiform activity, as well as apply a current to the seizure focus and arrest seizure progression.

### CROSS REFERENCE TO RELATED APPLICATIONS

This application claims the benefit of and priority to U.S. Provisional Parent Application Ser. No. 62/401,846, filed Sep. 29, 2016, the content of which is incorporated herein by reference herein in its entirety.

The present disclosure relates to generation, tracking, review and numerous aspects of the post-processing management of signals used to study, diagnose and treat neurological and psychiatric diseases, among other closely related aspects, including both novel and enhanced systems, devices and computer/processor based management of the same. To explain the present inventions, a survey of the state of the art both highlights the state of the art in this area, and shows the novelty of the instant contributions, it is respectfully proposed.

High-quality brain signals have been desiderata since cerebral vascular malformations have been noted and treated. The field comprising endovascular review, monitoring and navigation has quickly outmoded any type of scalp or even invasive or allopathic harvesting of neuro quality signals. As discussed in detail below, this makes it patent that the use of tools optimized for applications detailed herein presents patentable subject matters, being new, novel and non-obvious over existing systems, as detailed herein. Since this presents more than a mere step change within the field, it is important, ab initio, to set forth conditions under which the instant technology has evolved, to better understand just how different and better it appears to those practicing in this field then the current state of the art. In this light, nothing is meant to cast aspersions upon any of the prior art methods, techniques or approaches. They are included to inform the reader of the entire progression of this field, and provide a comprehensive survey of both the strategies and tactics used by others lo enable a fuller understanding of the instant systems.

Finally, it is also offered for consideration within the scope of the instant teachings, that optimizing of signal-to-noise ratios (and related properties) in application specific algorithms for specific disease states and treatments regimes likewise have provided additional subject matters believed to be within the scope of the instant teachings.

As noted above, the new paradigm comprising Endovascular Electroencephalography (EEG) enables numerous fields and approaches to treatment of the brain. In order to effectively manage the voluminous data presented over the past three to five years, five publications (listed hereafter and designated #1 to #5, along with being reproduced in their entirety in the Appendix, have been offered for consideration to show what the state of the art was just prior to the advent of the instant teachings), of which were published this year—2016 have been amalgamated and synthesized into the instant background sections. For ease of reference, please find [#1], [#2], [#3], [#4] and [#5] listed after each quotation, paraphrase and/or data set reproduced that was presented by others potentially outside of the scope of the public domain. Any error are the responsibility of the draftsman, not the authors of said papers.

Likewise, the full reference set is listed within the body of this document, with the order of the listing being the [Number] designated. The listing is offered for consideration after the section which it modifies, namely before the Detailed Description of the invention section, for ease of reference.

As previous investigators have explained and developed, namely to achieve intracranial electroencephalography (iEEG) limits the clinical application of sophisticated algorithms based on these electrodes to a subset of patients and experimental settings. [#1], [#2], [#5]

The literature is clear that—according to one study, for example, from 2004 to 2009 in the United States, a total of 101,123 patients were hospitalized for epilepsy. Of these patients, 40% (40,942) received scalp EEG monitoring and only 6% (6422) underwent intracranial EEG monitoring, with a comparable percentage of patients going on to epilepsy surgery. With the recent emergence of promising new surgical techniques for the management of epilepsy, access to definitive diagnosis through intracranial recording may represent a chokepoint in disseminating these compelling therapies. [#1], [#2] and [#5]

Intracranial electrodes, including subdural and depth electrodes, are employed in surgical planning when ambiguity remains with non-invasive methods (EEG, MEG, MRI, SPECT, PET). [#1], [#2], [#3], [#4] and [#5]

Further, direct Epilepsy applications include mapping of suspected medial temporal lobe epilepsy, stimulator implantation in subthalamic nucleus, and intraoperative functional mapping of language areas during tissue resection. Bypassing each of the layers of the scalp, skull, and dun, iEEG preserve): a wide range of frequency content (beyond 500 Hz), where spontaneous scalp EEG degrades above 50 Hz.

Source localization using subdural iEEG affords millimeter-scale spatial resolution, compared with centimeter-scale resolution using scalp EEG.

Despite these advantages, iEEG presents known challenges for the patient, tangible medical risks, and technical limitations. Considerable emotional stress and post-operative headache are routinely encountered with neurosurgical intervention. Depth electrodes involve drilling burr holes into the skull, and subdural grid electrodes typically require wider craniotomy. Intracranial bleed, infection, and edema along electrode tracts are known risks, with rates between 2% and 20% depending on the definition of complication. [#1], [#2], [#5]

For comparison, rates of complication with so called “Deep Brain Stimulation” or DBS electrodes are 3% for hemorrhage and 1% for infection, and as high as 10% if leads are temporarily externalized. Large subdural grids (>67 electrodes) are more prone to adverse events. The rates for depth electrodes are lower in comparison with subdural strip and grid electrodes. Repositioning grids or depth electrodes through revision surgery based on initial records is not practical. For select applications including mesial temporal sclerosis, foramen ovale electrodes provide intracranial recordings without craniotomy, but these recordings are restricted to the ambient cistern near the skull base, with risks including damage to the trigeminal nerve, infection, and bleeding.

Modern techniques allow physicians to safely traverse cerebral vessels to achieve a mil range of anatomical positions from the skull base to the cortical surface and throughout the brain. With roughly 800 neurointerventionalists across 400 hospitals in the United States, sub-millimeter guidewires and catheters are now routinely navigated through arterial and venous cerebral vasculature. Traditionally, advances using these techniques have focused almost exclusively on treating vascular pathology, including aneurysms, malformations, fistulas, and stroke. For many patients with these conditions, endovascular procedures have spared them open-skull surgery and prolonged hospital stay. The progressive refinement of these techniques has reduced the risk of intracranial complications (primarily related to stroke) to 3% from microcatheter intervention and 0.07% from diagnostic cerebral angiography. [#1], [#2], and [#5]

Unlike other disease states, those involving the brain do not translate well to small or even medium sized animal studies. Accordingly, previous demonstrations were performed in man, whereby clinically significant findings often lie, mostly below the surface of the skull, according to the teaching of the present inventions.

### BACKGROUND OF THE ART

The evolution of endovascular EEG recording and stimulation of the brain has been used for decades as a method to investigate neural function and treat conditions such as intractable epilepsy and Parkinson's disease. The current prior art surgical methods for accessing deep brain structures, however, require invasive open brain surgery: removing sections of skull in order to insert electrode arrays. While these invasive techniques have shown potential for deep brain stimulation (DBS) and brain-machine interlace (BMI) technologies, access to the deep brain structures requires the traumatic penetration of electrodes directly into brain tissue. Access to deep brain structures can also be achieved via minimally invasive techniques by utilizing the cerebrovascular system as a pathway, mapping element and finally as a therapy conduit. The measurement and recording of neural information from wires, catheters, and stent-like members, so called “stentrodes” and/or electrode arrays have demonstrated that the neural signals recorded from within a blood vessel are comparable to those obtained using invasive methods. This application set offer for consideration a bevy of applications derived from a detailed review of the landscape and adducing of a historical device perspective on the development of endovascular electroencephalography (EEG) and further discusses applications of endovascular EEG in minimally invasive neurosurgery in the fields of epilepsy, DBS, and BMI, among others. [#1], [#2], [#3], [#4] and [#5]

Accordingly, it is respectfully submitted that the instant teachings, both for identifying an unresolved set of issues and suggesting numerous solutions constitutes progress in science and the useful art to be recognized by US Letters Patent, inter alia.

### OBJECTS AND SUMMARY OF THE INVENTION

Briefly stated, minimally invasive systems used novel enhanced intracranial signals to develop, characterize and ameliorate challenges and disease states with application specific tools modified from tradition EEG for Epilepsy to address a myriad of conditions in patients, without any need for invasive protocols traditionally employed.

According to embodiments there is provided a microwire with multiple recording leads, comprising in combination, at least about 200 cm of length; having zones of flexure allowing for navigation through tortuous cerebrovascular circulatory pathways, with a low profile of under 0.0165 inches, further comprising an insulated coating around a metal wire, with gaps disposed thereupon at locations of each respective recording lead; and the microwire system is deliverable endovascularly and MRI compatible.

According to embodiments there is provided an improved device that can be unsheathed which produces a fanlike array of leads, further comprising: at least about 180 cm of length; changing from a first to a second position upon desired triggering and release, in situ; the device having appropriate density and modulus to promote navigation through tortuous cerebrovascular circulatory pathways; having of profile of less than at least about 0.028 inches.

According to embodiments there is provided a novel stent-like device having multiple recording leads, comprising, in combination; at least a first scaffolding structure, effective for being navigated with an endovascular delivery system, through a low profile introducing means; a plurality of recording leads, which leads are disposed flexibly enough to be delivered unscathed to a target situs; an open or closed cell structure; radiopacity trackability and self-expansion, whereby the stent-like device transforms from a first to a second position, without compromise to the subject recording leads and a ratio of Hoop Strength to Chronic Outward Radial force enabling it to be taken from a first to a second position, within or without a sheath, whereby the device car support a set of recording leads, electrodes or the like assemblies to sense, record, transmit and interpret data, including any specialized chip-sets, processors or general or special purpose computing toots in hardware, software or cloud-rink and enabled form.

According to embodiments there is provided A method for treating assessing, treating, ameliorating or otherwise addressing Epilepsy, comprising, in combination; providing at least a tool as described in claims 1-10 with multiple recording leads: targeting select regions and tissue sites for measurement, harvesting and recording of neural information at least a first means for interpreting select aspects of the harvested neural information, at least a second means for generating an appropriate signal response to select aspects of the harvested neural information, and, delivery means for directing the signals toward pre-selected or ad-hoc chosen regions and tissue sites.

According to embodiments, there is provided an improved system for generating and managing intracranial brain signals, which comprises, in combination at least a device, tool or instrument defined herein or later developed having multiple recording leads, sensors, arrays, panels and/or means for generating and interpreting signals, an insertion and removal mechanism; and a complementary or supplemental or master processor or computer means for storing, arraying and transmitting signals, responsive to commands of a user, whereby signal detection, review and analysis is performed and data generated and relied upon for further diagnosis and treatment.

According to embodiments there is provided an improved system, according and including any devices and methods of those claims which can be permanently implanted (like a pacemaker) that can both sense epileptiform activity, as well as apply a current to the seizure focus and arrest seizure progression, which does not require craniotomy and direct cortical placement of electrodes.

According lo embodiment there is are provided devices and systems or methods of the present invention as disclosed and claimed in claims 1-16 herein or in materials incorporated by reference wherein a safety profile shall be determined for the endovascular ablation of seizure foci, endovascular stimulation in DBS, and the stenting, with and without recording leads or arrays or multiple arrays of electrodes, sensors and the like signal harvesting, processing and storage means.

According to embodiments there are provided devices, methods, systems, including delivery systems all disclosed and claimed herein, and incorporated by reference herein employed for the surgical treatment of epileptogenic foci via endovascular ablation.

According to embodiments there is provided use of endovascular EEG in the preoperative evaluation of patients for epilepsy surgery, in complement with the determination of resection margins that provide the clinically optimal targets to be treated endovascularly, according to any of the disclosures, devices, systems, methods, strategies and teachings express and implied of the instant application for US Letters Patent.

The purpose of these inventions is to develop endovascular techniques to detect cerebral electrical signals (EEG) for both diagnostic and therapeutic purposes. There are significant limitations to the most commonly used EEG techniques, which use scalp electrodes. For accurate cortical mapping, surgical craniotomy with placement of electrode grids directly on the cortical surface is often required. We have access to these same locations however with endovascular catheters with relative ease, all the while remaining minimally invasive.

A salient proof-of-principal experiment to demonstrate that we can record EEG signals with endovascular catheters has been managed. To do this, we used of off-the-shelf cardiac EP catheters, which offer the advantage of multiple recording electrodes in a variety of configurations (up to 20 electrodes per catheter).

We then proceeded to record signals, apply a stimulus, and then detect the EEG change. While there has been some previous work in this area, successfully performing this experiment, in and of itself by recognizing the problem—eg. The need for better quality brain signals driven by EE, addresses plethoric needs, and constitutes both progress in science and the useful arts, and it is respectfully submitted patentable subject matter.

The final result is a system that can be permanently implanted (like a pacemaker) that can both sense epileptiform activity, as well as apply a current to the seizure focus and arrest seizure progression. There are some devices currently in the market that are able to do this, but again, require craniotomy and direct conical placement of electrodes.

### EXAMPLES FIRST

According to live instant teachings embodiments include, a microwire with electrical insulation that allows for multiple recording channels along the length of the wire; a catheter with multiple recording channels along its length, and a stent-shaped device with multiple recording electrodes along its length.

These inventions work because they are driven by concepts for vascular access including the end target will be the cerebral arteries, veins, subarachnoid, and subdural spaces; routes to access these territories include through the arteries and veins of the leg, the arm, neck, and the face. For long term implantations, the devices can be tunneled through the skin to minimize infection risk and allow more comfort to the patients.

Data Analysis drive by applications has proven that signal analysis needs to take into account pulsation artifact and other artifacts associated with movement of the recording device. A method to account for these artifacts is to adjust the signal for the artifacts based on a lead that measures cardiac activity (i.e. an electrocardiography lead).

What has been achieved is the signal analysis algorithm is able to produce a clean tracing of cerebral electrical activity, which can then be processed for automatic detection of the queried neuronal activity (such as epileptiform activity).

### BRIEF DESCRIPTION OF THE DRAWINGS

Various preferred embodiments are described herein with references to the drawings in which merely illustrative views are offered for consideration, whereby:

FIG. 1 is a microwire with multiple recording leads;

FIG. 2 shows a microcatheter with multiple recording leads;

FIG. 3 shows a device that can be unsheathed that produces a fanlike array of leads;

FIG. 4 depicts a stent-like device with multiple recording leads; and

FIG. 5 illustrates schematically a battery pack like device that can connect to these devices.

Although mentioned above, it is believed that further review of the evolution of this field is instructive in grasping the improvements provided by the instant systems.

Those skilled in the art understand how endovascular techniques facilitate such improvement given published data such as using clinical epileptic crisis as the standard reference, the following statistical parameters were determined to describe intra arterial EEG: sensitivity, 93.33%; specificity, 80%; positive predictive value, 82.35%; and negative predictive value, 92.3%. [#2]

Mikuni et al. demonstrated a predictive relationship between cavernous sinus recordings, revealing undetected EEG measurements by extracranial recordings and the presence of epileptogenic foci in the mesial temporal lobe as opposed to the lateral temporal lobe. In these experiments, this group secured the wire to the cervical skin, and for the first time, continued recording EEG for 3 to 40 hours following angiography (n=5). [#1], [#2], and [#5]

Kunieda et al. expanded on the work of Mikuni et al. in terms of the detection of endovascular EEG from the cavernous sinus and the superior petrosal sinus and the length of the recording time. Kunieda et al. used a stainless steel Seeker Lite-10 wire (Target) coated in polymer with a single platinum electrode and detected 80% of ictal foci in patients (n=5) with wires implanted for 4 to 75 hours. As mentioned in previous studies, this group reported pulse artifacts in the recordings. The work by Kunieda et al. was limited by the patients' movements in the post anesthesia monitoring period, as patients were at risk of both wire breakage and inferior recordings. There were significant advances in endovascular wire recordings from the early 1970s through the late 1990s. At this point in time, recordings were usually from a single unipolar electrode, although the first endovascular EEG recording employed 2 electrodes for bipolar recording. Experimentation was translated from baboons to humans, and the sample sizes increased from the single recording in humans in 1973 to recordings in 30 patients in 1999. From the first endovascular EEG recording in 1973 there were also significant advances in both the ability to record from a venous approach and continue recordings outside of the angiography procedure. [#1], [#2], [#4] and [#5]

Catheter Recordings have Shown Promise Vet Need to be Optimized

In addition to the increasing miniaturization of wire electrodes in 1998, a new technology for recording endovascular EEG was developed at the same time: the catheter electrode recording device. Thömke et al. demonstrated that a catheter (Pathfinder (R) with 8 electrode pairs, which could simultaneously record from 16 locations in the brain (within a 72-mm distance), could be used to record endovascular EEG in humans.

Bower et al. advanced the catheter recording technique in 2013 with the inclusion of more electrodes and the first venous catheter recording. This group used 16 microelectrodes contained within a 4-contact depth electrode for the intravenous recording of EEG changes induced by penicillin and cortical electrical stimulation in pigs that underwent craniectomy for catheter placement in the superior sagittal sinus. The intravenous recordings were consistent in amplitude with simultaneously recorded subdural electrodes, and the intravascular method was successful in providing the location of seizure activity. Although Bower et al advanced the catheter technique for its use in the superior sagittal sinus, the use of craniectomy for catheter placement limits the clinical utility of this demonstration as a future minimally invasive therapy. Prior to the advent of the instant teachings. [#1], [#2], [#4] and [#5]

### STENTRODE™ Recordings

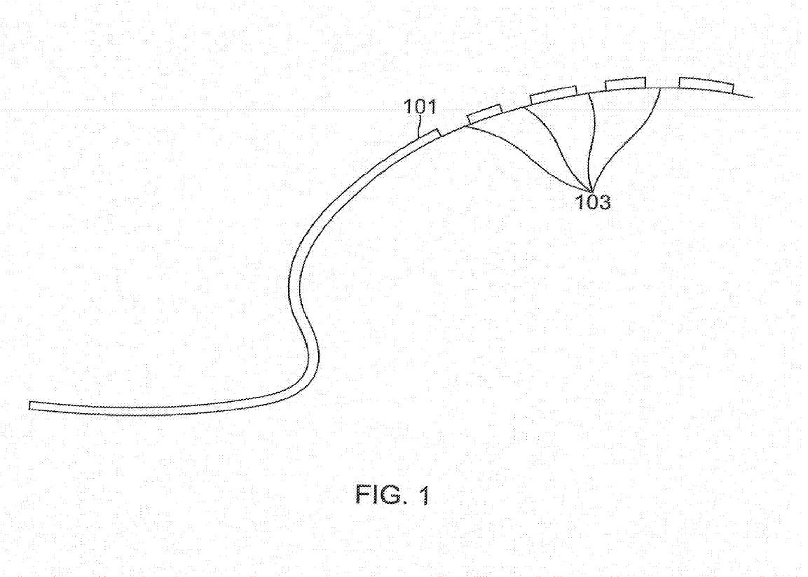

Endovascular EEG recording technology was advanced in terms of device design, location of device deployment, and the ability to chronically record endovascular EEG in 2016 with the development of the stentrode, which it pictured in FIG. 1. Oxley et al. determined through MRI analysis of 50 patients that the human superficial cortical veins and superior sagittal sinus, with intraluminal diameters of 2 to 8 mm, were sufficient conduits for measuring neural activity from the sensorimotor cortex. Since the superior sagittal sinus in sheep is comparable to the central sulcus vein in humans, sheep were used to develop the animal model. Oxley et al. used a self-expanding stentrode array that was implanted in a superficial cortical vein via catheter angiography in order to chronically record neural activity (recordings up to 190 days) from the motor cortex in sheep and compared the recordings to both subdural and epidural surface arrays. The endovascular approach was comparable to the performance of the epidural array, but the performance of the subdural array was slightly superior. A pilot study resulted in the development of an epileptic seizure that was overcome by intravenous diazepam in 1 sheep, and repetitive neck movements by the freely moving sheep resulted in wire fatigue. There were also chewing muscle artifacts present in the recordings. Unlike the superior sagittal sinus recordings obtained by Bower et al., the animals in the experiments conducted by Oxley et al. did not undergo craniectomy, thereby increasing the clinical utility of this approach as a minimally invasive therapy. Furthermore, the demonstration of the ability to chronically record endovascular EEG in the proximity of the sensorimotor cortex allows for possible applications in DBS and BMI, although further investigations of chronic recordings from endovascular stentrodes in humans are needed.

Endovascular recording devices have advanced from wire recordings to micro/nanowire recordings, to catheter recordings, and most recently stentrode recordings. Electrode arrays were developed from the increasing miniaturization in recording wires and electrodes and the development of catheter and stem-electrode recording technology. The advances in endovascular recording, have made it possible to obtain increasing amount of information about neural activity from the endovascular environment. Recording sites lave also increased with the ability to record from the venous system. Since the superior sagittal sinus is located superficial to the sensorimotor cortex, and the ability to chronically record endovascular EEG in freely moving animals has been demonstrated, there are possible future applications of the endovascular approach to EEG in BMI. The endovascular approach to EEG recording also promises greater safety for neural interfaces, as evidenced by the work of Garcia-Asensio et al. who, with the largest human sample size in this review, reported no side effects or adverse events in their patients with up to 3 years of follow-up. In contrast, the subdural grid electrodes, to which many of the reviewed studies compared their endovascular recordings, were shown by Hamer et al. to have a complication rate of 26.3% (52 of 198 monitoring sessions). [#2] and [#5]

While endovascular EEG offers an advantageous safely profile, risks should not be overlooked, such as the transient, but tolerable, headaches and retro bulbar pain as reported in the experiments of Mikuni et al. (such headaches were excluded from the study by Hamer et al.)

### Epilepsy

As previously discussed, the tools for the application of an endovascular approach to the recording of EEG are numerous in the field of epilepsy surgery. Current evaluative methods that are efficacious for the detection of seizure foci employ techniques such as the use of intracranial epidural arrays: however, this approach requires craniotomy. Likewise, intracranial depth electrode implantation aids in the detection of epileptogenic focus surgical margins, but requires invasive bur holes. The endovascular approach could be applied preoperatively for the minimally invasive localization of seizure foci and possibly the determination of resection margins.

It is likewise proposed herein that the endovascular approach can be employed for the surgical treatment of epileptogenic foci via endovascular ablation, as Ammerman et al. described a case report of a patient who became seizure free while receiving antiepileptic drugs following a stroke in the territory of the anterior choroidal artery, most likely due to catheter emboli following endovascular Wada testing.

According to the instant teachings, it is further proposed that that DBS can be performed using un endovascular approach. Teplitzky et al. demonstrated the feasibility of an endovascular approach for DBS via computational modeling. This group identified 5 DBS targets with adjacent vasculatures that were at least 1 mm in intraluminal diameter (anterior nucleus of the thalamus, fornix, nucleus accumbens, subgenual cingulate white matter, and ventral capsule) by modeling the cerebrovascular system. The subgenual cingulate white mailer and fornix were further investigated as potential endovascular DBS targets (which were cited to have roles in depression and memory disorders, respectively), and modeling determined that a ring electrode was preferred over a guidewire electrode for endovascular DBS (due to enhanced vessel wall anchoring capabilities, decreased distance from the electrode to the DBS target, and enhanced neural activation). Teplitzky et al. also demonstrated that with a unilateral electrode implant, endovascular DBS was superior to stereotactic DBS in the production of contralateral activation and comparable to stereotactic DBS in neuronal activation. Further investigation into the stimulation parameters (such as the current levels) and the safety profile of intravascular stimulation is necessary. [#1], [#2], [#3], [#4] and [#5]

Others have also shown that in terms of Brain-Machine Interfaces the endovascular approach is promising as a chronic and minimally invasive technique to achieving a BMI. Despite the advantages of an invasive approach to BMI (electrocorticography) over scalp EEG (e.g., higher signal bandwidth, closer location to the recording target, higher spatial resolution and signal amplitude, and the lack of interference from both etectrooculography and electromyography [with the exception of the reference electrode]), an invasive approach to BMI requires the implantation of a foreign body into the brain parenchyma, which may result in inflammation. With an increase in the cross-sectional area of the device, there is increased inflammation in the week following implantation (most likely due to increased parenchymal damage with insertion). Furthermore, chronic inflammation in the 6 weeks following implantation is independent of device size, and it is believed that increasingly small devices will not circumvent inflammation in its entirety. An endovascular approach may remedy this problem, as trauma to the brain parenchyma is not associated with electrode implantation. [#1], [#2], [#3], [#4] and [#5]

According to the instant teachings, endovascular approaches to, are advantageous over current invasive approaches. Invasive intracranial subdural electrodes are limited to recording only in the space over which they are implanted (which may lead to limited analyses). The cerebrovascular system, however, provides a minimally invasive channel to the area superficial to the sensorimotor cortex, allowing for an endovascular minimally invasive approach to BMI.

It has therefore been proposed, and it is respectfully submitted shown by the instant teachings that, in neurosurgery, minimally invasive endovascular techniques have become a tool for the evaluation of epileptogenic foci. In cardiology, catheter ablation proved to be efficacious in curing cardiac arrhythmia, and it has now been claimed herein that catheter ablation can be supported by the instant tools in epilepsy surgery.

Similarity disclosed and claimed herein are the neuro analogues to the development of smaller, chronically implanted cardiac defibrillators that were efficacious for treating ventricular fibrillation and ventricular tachycardia became the standard in cardiac electrophysiology; in the same way, chronically implanted electrodes for the prediction and treatment of epilepsy that were deposited endovascularly may become a tool in neurosurgery. An intravenous vagal nerve stimulator placed in the superior vena cava is currently being investigated in cardiology. This device has been shown to decrease left ventricular end-diastolic pressure, decrease the size of an infarction, increase left ventricular ejection fraction at 1 month following induced coronary ischemia, and prevent ischemia-induced ventricular arrhythmias in dogs. Furthermore, the efficacy of an intravenous phrenic nerve stimulator for the treatment of patients with central sleep apnea is currently being investigated in a randomized controlled trial, and a prior nonrandomized study showed a reduction in the apnea-hypopnea index scores by 55% at 3 months after the initiation of treatment. Similar advances in neural endovascular stimulation could lead to further advancements in epilepsy management, DBS, and BMI applications. Endovascular recording technology has advanced from the first wire recording in 1973, to the development of microwire and nanowire recordings in 1998 and 2005, respectively, catheter recordings in 1998, and the stentrode in 2015. With advances in device technology, there was a transition from the use of single unipolar electrodes to the use of electrode arrays. [#2] and [#5]

According to the present inventor and colleagues, endovascular EEG can be used in the preoperative evaluation of patients for epilepsy surgery, or even in the determination of resection margins that could possibly be treated endovascularly. In addition, computational modeling has demonstrated the feasibility of an endovascular approach to DBS, and the ability to chronically record in the superior sagittal sinus superficial to the sensorimotor cortex may lead to the achievement of a minimally invasive BMI.

In order for the use of endovascular EEG to be translated from research to clinical practice, the use of the tools of the present invention are is required. One can learn from the literature that the safety profile will need to be determined for the endovascular ablation of seizure foci, endovascular stimulation in DBS, and the stenting of the superior sagittal sinus for BMI. In addition, further research has been done to invent retrievable neural endovascular recording devices that would both mitigate the risk of venous infarction and stroke and eliminate the need for chronic anticoagulation. Stimulation parameters will are being determined to achieve minimally invasive DBS, and the maximum number of electrodes that can be used to achieve minimally invasive BMI will need to be determined. [#1], [#2], [190 3], [#4] and [#5]

### CITED REFERENCES LISTED IN THE BACKGROUND BY NUMBER ORDER #1-#5

Signal quality of endovascular electroencephalography. J Neural Eng. 2016 February: 13(1):016016. doi: 10.1088/1741-2560/13/1/016016. Epub 2016 Jan. 6. [1]
The evolution of endovascular electroencephalography: historical perspective and future applications. Neurosurg Focus. 2016 May; 40(5):E7. doi: 10.3171/2016.3.FOCUS156355.[2]
Endovascular electroencephalography during an intracarotid amobarbital test with simultaneous recordings from 16 electrodes. J Neurol Neurosurg Psychiatry 1998; 64:565 doi:10.1136/jnnp.64.4.565. [3]
Endovascular electroencephalography: the technique and its application during carotid amytal assessment. J Neurol Neurosurg Psychiatry. 1997 February; 62(2):193-195. [4]
Minimally invasive endovascular stent-electrode array for high-fidelity, chronic recordings of cortical neural activity. Nature Biotechnology 34, 320-327 (2016) doi: 10.1038/nbt.3428

### DETAILED DESCRIPTION OF THE INVENTIONS

The following US Letters Patents are expressly incorporated by reference: U.S. Pat. Nos. 8,066,757; 8,088,140; 8,585,713; 8,926,680 Each of which is expressly incorporated by reference herein, as if fully set forth, these patents represent the state of the art in Neurovascular access and stenting, include technology developed by MINDFRAME®, INC (acquired by COVIDIEN in 2011). Likewise, each of said references (authored by the instant draftsman) has been reviewed again in detail and are each clearly distinguished form the instant teachings.

The present inventors have discovered that they can develop endovascular techniques to detect cerebral electrical signals (EEG) for both diagnostic and therapeutic purposes. With an array of basic tools, they offer for consideration novel and enhanced approaches to treating challenges within the brain.

As discussed, there are significant limitations to the most commonly used EEG techniques, which use scalp electrodes. For accurate cortical mapping, surgical craniotomy with placement of electrode grids directly on the cortical surface is often required. We can access these same locations however with endovascular catheters with relative ease, all the while remaining minimally invasive.

Composed variously of platinum and nitinol alloys, the guidewires that facilitate endovascular access are conductive, atraumatic, biologically inert and torqueable. When passed into the cerebral vasculature of the human brain, these guidewires record evoked potentials with substantially larger magnitude than scalp potentials. Guidewires have been left within venous sinuses for prolonged recording in an epilepsy monitoring unit. Recent animal models have reproduced these findings with platinum electrodes.

Referring now to FIG. 1, there is shown a microwire 101, with multiple recording leads 103. As known to those of skill in the art, devices up to and over 200 cm in length can be emplaced within the cerebral vasculature, with and without other devices, according to the instant teachings. As discussed in the claims and shown in the figure said novel enhanced microwire with multiple recording leads, functions as expected to effectuate application specific protocols, the device composing in combination; at least about 200 cm of length, the microwire having zones of flexure allowing for navigation through tortuous cerebrovascular circulatory pathways; with a low profile of under 0.0165 inches, further comprising an insulated coating around a metal wire, with gaps disposed thereupon at locations of each respective recording lead.

The present inventor knows the instant system has utility in epilepsy because of the literature in combination with the prototypes of the instant system in process. For example, it has been reported that, in one study, platinum electrode strips were surgically placed in the superior sagittal sinus of sheep to record penicillin-induced ictal waveforms. Similar unpublished work has been performed by others. In the aforementioned studies, signal amplitudes resembled those of subdural iEEG. In contrast, evoked potentials recorded from peripheral nerves are comparable in amplitude between endovascular and skin-surface recordings, where interposed skull is not present to impede dermal EEG (Llinas et al 2005). [#1], [#2] and [#5]

Despite this literature on endovascular EEG, the fundamental question of signal quality remains unanswered. This is because signal amplitude alone does not determine the basic measures of signal quality that are important to iEEG applications, including signal-to-noise ratio (SNR), frequency content, and spatial sensitivity. With regards to signal variability (in terms of SNR), there are several practical reasons for concern. Mechanical pulsation artifact is known to degrade subdural electrodes that are located in proximity to cortical arteries, and this effect could be magnified within the vessel. Cardiac field artifact in subdural electrodes could be more pronounced with guidewire electrodes that pass through the chest cavity en-route to the brain. Scalp EEG and transcranial iEEG leads are kept short to avoid electrical interference, but guide wires are nearly two orders of magnitude longer at 175 lo 200 cm. With regards to frequency content, the impedance spectra of guidewire metal alloys are uncharacterized, where Ag/AgCl and platinum are proven electrode materials applied in scalp EEG and subdural iEEG respectively. [#1], [#2], [190 3], [#4] and [#5]

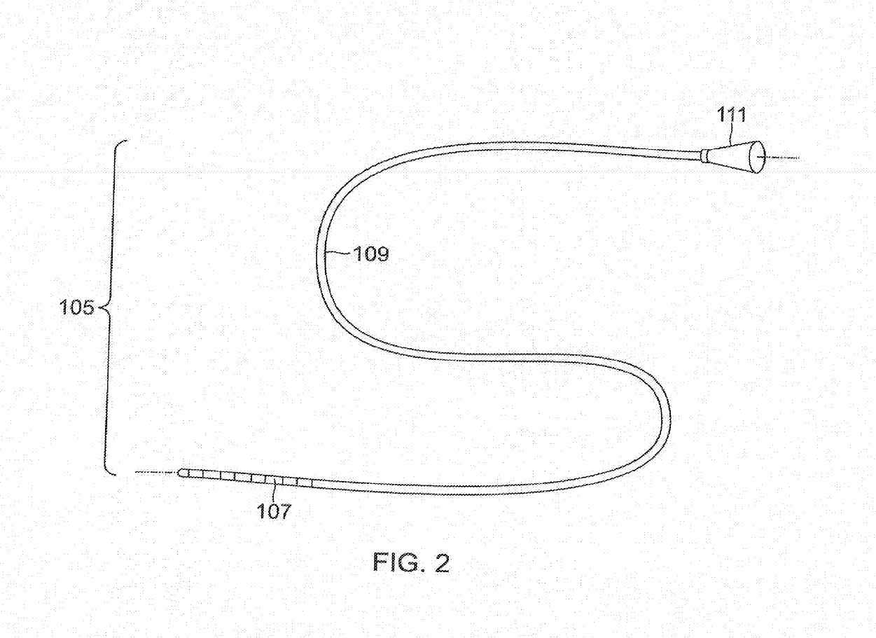

Referring now to FIG. 2 which shows a microcatheter with multiple recording leads 105, the body of the catheter having recording leads or recording lead array 107 shown at the distal end of catheter body 109, the proximate end including a port 111, for mating with the balance of a claimed procedure set and delivery system.

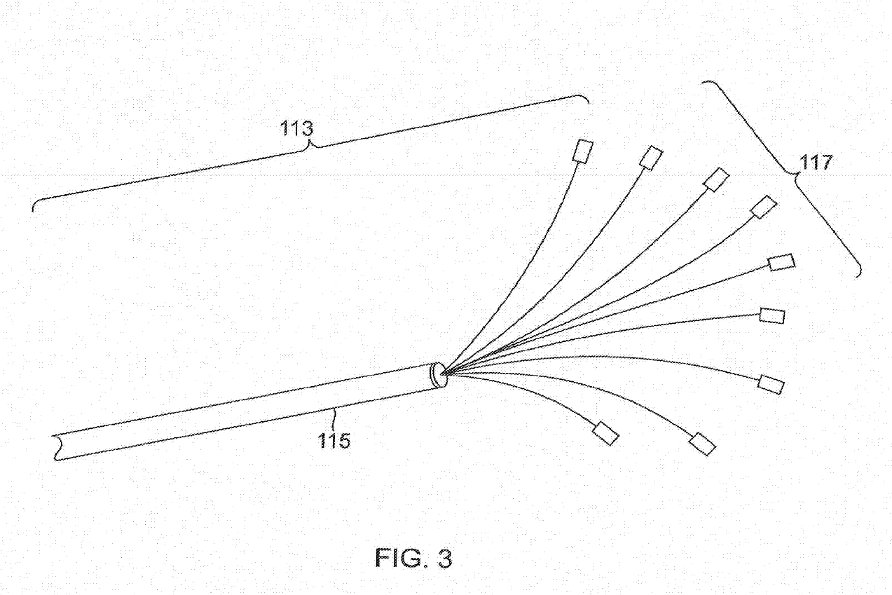

FIG. 3 shows a device 113 that can be unsheathed which produces a fanlike array of lead, this device 113 includes a device body 115 and the fanlike array of leads 117. Those skilled in the art know that such a device is used, depending on the procedure, with other microcatheter sets and tools to be part of an overall approach to sense deliver and retrieve signals.

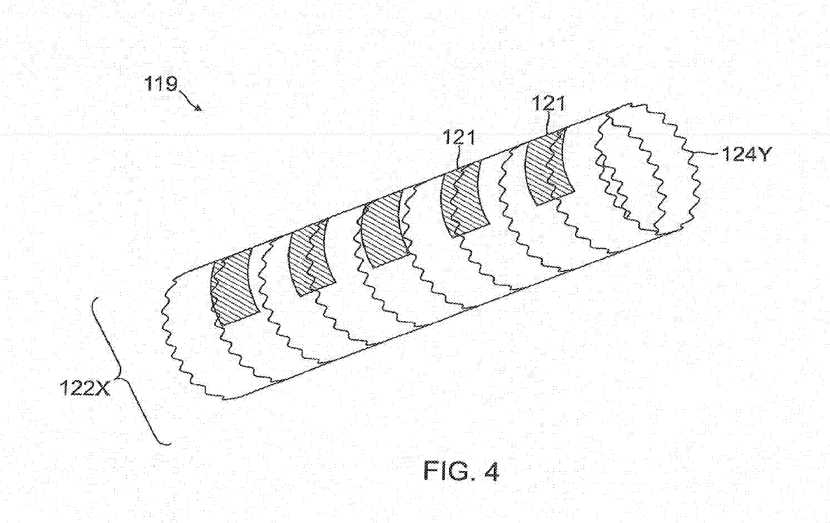

FIG. 4 depicts a stent-like device 119 with multiple recording leads 121. Those skilled in the art understand that such a novel, stent-like device, having multiple recording leads, comprising, in combination, at least a first scaffolding structure, effective for being navigated with an endovascular delivery system, through a low profile introducing means, a plurality of recording leads, which leads are disposed flexibly enough to be delivered unscathed to a target situs; an open or closed cell structure, radiopacity, trackability and self-expansion, whereby the stent-like device transforms from a first to a second position, without compromise to the subject recording leads, is driven by the ratio of 122X or the hoop strength (HS), v. the chrome outward radial force (CORF) 124Y.

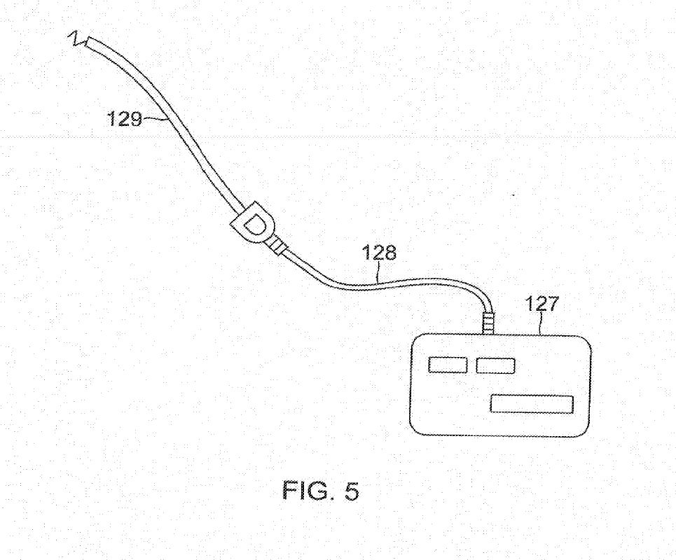

Finally, FIG. 5 illustrates schematically a battery pack like device 127 that can connect to intracranial recording devices/and-or wirelessly do so. It is known to place such devices with the subcutaneous tissue and communicate with handheld person digital assistants, databases and health care services.

It is further known to tunnel connections under the skin to the intracranial devices, and the device and entire system remain MRI compatible.

Computer technology and specialized computer devices, hardware and software, including applications, new or old devices and new chip-sets are all within the ambit and scope of the instant teachings. For example, if there is smart phone technology that supports an application running on general or special purpose computer and stored in a database, using information generated by the instant system, it is known to those skilled in the art that, outside of artifacts, the data and systems for generating the same are proprietary and expressly included within this patent application.

Unless otherwise indicated, all numbers expressing quantities of ingredients, properties such as molecular weight, reaction conditions, and so forth used in the specification and claims are in be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed clement essential to the practice of the invention.

Groupings of alternative elements or embodiments of the invention disclosed herein ate not to be construed as limitations. Each group member may be referred lo and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes toy clement, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. An enhanced microwire system with multiple recording leads, comprising in combination:
at least about 200 cm of length;
the micro wire system having zones of flexure allowing for navigation through tortuous cerebrovascular circulatory pathways;
with a low profile of under 0.0165 inches (1.5 F);
further comprising an insulated coating around a metal wire, with gaps disposed thereupon at locations of each respective recording lead.

2. The enhanced microwire system of claim 1, deliverable endovascularly and MRI compatible.

3. A microcatheter having multiple recording leads, comprising:
at least about 180 cm of length;
the microcatheter having appropriate density and modulus to promote navigation through tortuous cerebrovascular circulator) pathways;
having of profile of less than approximately 0.028 inches (2 F).

4. The microcatheter of claim 3, further comprising a hollow-bore permitting over the microwire (OTW) navigation.

5. The microcatheter of claim 4, deliverable endovascularly and MRI compatible.

6. An improved device that can be unsheathed which produces a fanlike array of leads, further comprising:
at least about 180 cm of length;
changing from a first to a second position upon desired triggering and release, in situ;
the device having appropriate density and modulus to promote navigation through tortuous cerebrovascular circulatory pathways;
having of profile of less than at least about 0.028 inches (2 F).

7. The improved device of claim 6, further comprising a hollow-bore permitting over the microwire (OTW) navigation.

8. The improved device of claim 7, deliverable endovascularly and MRI compatible.

9. A novel stent-like device having multiple recording leads, composing, in combination:
At least a first scaffolding structure, effective for being navigated with an endovascular delivery system, through a low profile introducing means;
a plurality of recording leads, which leads are disposed flexibly enough to be delivered unscathed to a target situs;
an open or closed cell structure;
radiopacity, trackability and self-expansion, whereby the stent-like device transforms from a first to a second position, without compromise to the subject recording leads.

10. The novel stem-like device further comprising:
a ratio of Hoop Strength to Chronic Outward Radial force enabling it to be taken from a first lo a second position, within or without a sheath, whereby the device can support a set of recording leads, electrodes or the like assemblies to sense, record, transmit and interpret data, including any specialized chip-sets, processors or general or special purpose computing tools in hardware, software or cloud-link and enabled form.

11. An improved system for generating and managing intracranial brain signals, which comprises, in combination:
at least a device, tool or instrument defined herein having multiple recording leads, sensors, arrays, panels and/or means for generating and interpreting signals;
an insertion and removal mechanism; and
a complementary or supplemental or master processor or computer means for storing, arraying and transmitting signals, responsive to commands of a user, whereby signal detection, review and analysis is performed and data generated and relied upon for further diagnosis and treatment.

12. An improved system, according to claim 7, and including any devices which can be permanently implanted (like a pacemaker) that can both sense epileptiform activity, as well as apply a current to the seizure focus and arrest seizure progression, which does not require craniotomy and direct cortical placement of electrodes.

13. An improved system, according to claim 12, wherein a safety profile shall be determined for the endovascular ablation of seizure foci, endovascular stimulation in DBS, and the stenting, with and without recording leads or arrays or multiple arrays of electrodes, sensors and the like signal harvesting, processing and storage means.

14. An improved system, according to claim 13, whereby use of endovascular EEG in the preoperative evaluation of patients for epilepsy surgery is done diagnostically, in complement with the determination of resection margins that provide the clinically optimal targets to be treated endovascularly.
